(19) United States
(12) Patent Application Publication
(10) Pub. No.: US 2023/0201276 A1
(43) Pub. Date: Jun. 29, 2023

(54) METHOD OF TREATING OR PREVENTING AN INFECTION

(71) Applicant: SUPERBREWED FOOD, INC., New Castle, DE (US)

(72) Inventors: Alon KARPOL, Tel-Mond (IL); Hadar GILARY, Kibbutz Magal (IL); Shawn William JONES, Bear, DE (US); Bryan P. TRACY, Wilmington, DE (US); Biniam MARU, Bear, DE (US); Aharon M. EYAL, Jerusalem (IL); Sasson SOMEKH, Los Altos Hills, CA (US)

(21) Appl. No.: 17/926,603

(22) PCT Filed: May 21, 2021
(86) PCT No.: PCT/IB2021/054408

§ 371 Date: Nov. 20, 2022

Related U.S. Application Data
(60) Provisional application No. 63/028,298, filed on May 21, 2020.

(51) Int. Cl. A61K 35/742; A61K 35/747; A61K 35/744; A61K 35/745; A61K 35/741

(52) U.S. Cl. CPC A61K 35/742; A61K 35/747; A61K 35/744; A61K 35/745; A61K 35/741

(57) ABSTRACT

Provided are compositions comprising at least one organism of a strain of the order Clostridiales and methods of use thereof for protecting against an infection, such as a viral, protozoal or fungal infection in an animal. Also provided are methods for improving hatchability of an egg under contaminated conditions by protecting against infection of an avian in ovo.

### FIELD OF THE INVENTION

The field of art to which this invention generally pertains is immunoprotection of an animal and more specifically to methods of treating or preventing an infection, such as a viral, protozoal or fungal infection by use of compositions comprising cells of an organism of the order Clostridiales.

### BACKGROUND OF THE INVENTION

A number of documents have appeared in the literature describing the scientific basis for use of probiotics, as intestinal inoculants for animals. Since Metchnikoffs work in the 19^{th} century, which first established the probiotic understanding as we know it, many studies have shown the ability of microorganisms to suppress pathogen growth, improve feed conversion ratio or stimulate the immune system.

It is generally held that during periods of low disease resistance, such as stress, undesirable microorganisms are able to proliferate in the gastrointestinal tract (GI tract) of animals, humans included. Maintaining a normal, healthy balance of microorganisms is deemed to be critical, particularly during such stressful periods. The concept underlying use of probiotics therefore is that, if sufficient numbers of an appropriate microorganism(s) are introduced into the intestinal tract (i) at times of stress and/or disease, (ii) at birth, or (iii) after antibiotic treatment, the negative consequences of the microbial imbalances (Dysbiosis) can be minimized or overcome. Using such preparations of live, naturally occurring microorganisms helps restore and maintain the proper balance of beneficial microbes in the GI tract during times of stress, disease, and following antibiotic therapy.

The main bacteria that are currently being administered as probiotics are genera such as Lactobacillus, Enterococcus, and Bifidobacterium.

### SUMMARY OF THE INVENTION

According to an aspect of some embodiments of the present invention, there is provided a method of treating or preventing an infection in a subject in need thereof, the method comprising administering to the subject a composition comprising at least one organism of a strain of the order Clostridiales to an animal in need thereof.

According to an aspect of some embodiments of the present invention, there is provided a composition comprising at least one organism of a strain of the order Clostridiales for use in treating or preventing an infection.

### DETAILED DESCRIPTION OF THE INVENTION

The present invention relates to of treating an infection, such as a viral, protozoal or fungal infection by use of compositions comprising cells of an organism of the order Clostridiales.

The present inventors have surprisingly found that administration of a composition comprising cells of at least one strain of an organism of the order Clostridiales are useful in treating a viral infection or symptoms thereof.

Without wishing to be bound by any one theory, the present inventors hypothesize that the Clostridiale microorganisms administered to an animal colonize the digestive system of the animal, including the gut the cera, ilium, duodenum, jejunum. After colonization the microorganism begin to produce short-chain fatty acids (SCFAs) such as butyric acid, propionic acid, valeric acid and acetic acid, which are absorbed by the epithelial cells of the animal, serving as an energy source for those cells.

Since epithelial cells constitute part of the innate immune system of an animal, which provides a broad spectrum, first line of defense of the animal against pathogens, such as pathogenic bacteria, protozoa, fungus and viruses, a composition which provides an energy source to the epithelial cells and therefore indirectly activates such cells, may potentially be useful in stimulation of an innate immune response, such as against a virus, protozoa or fungus.

Furthermore, epithelial cells are responsible for the generation of the mucin layer, secretion of antimicrobial proteins, transport of luminal antigens to dendritic cells, which act as messengers between the innate and the adaptive immune systems. By sampling the luminal space, those cells can present foreign antigens such as viral particles to the adaptive immune respond, helping to increase the adaptive respond against viruses (such as SARS-Cov-2).

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

As used herein, the term “immunoprotection” is intended to mean protection against detrimental effects of an antigen. The composition providing immunoprotection to a subject may be administered prior to, subsequent to or substantially simultaneously with exposure to the antigen.

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

As used herein, the term “colonizing” with regard to a digestive tract of an animal is intended to mean populating the digestive tract with at least 10\hat{ }2 CFU of bacteria per 1 g.

As used herein, the term “regulating the immune system” is intended to mean stimulating or inhibiting activity of the immune system as appropriate for improving the health of the animal, such as by increasing immune activity against a pathogen.

As used herein, the term “tolerance to pH” refers to survival of at least 1% of the cells exposed to the condition for duration of 2 hours.

As used herein, the term “tolerance to bile salts” with regard to cells refers to survival of at least 10% of the cells when exposed to at a solution comprising least 0.2wt % bile salts, at least 1 wt % bile salts or even at least 2 wt % bile salts for a duration of 24 hours.

According to an aspect of an embodiment of the present invention, there is provided a method of treating or preventing an infection in a subject in need thereof, the method comprising administering to the subject a composition comprising at least one organism a strain of the order Clostridiales.

According to an aspect of some embodiments of the present invention, there is provided a composition comprising at least one organism of a strain of the order Clostridiales for use in treating or preventing an infection.

According to an embodiment, the infection is selected from the group consisting of a viral infection, a protozoal infection and a fungal infection.

According to an embodiment, the infection is a viral infection.

According to an embodiment, the viral infection is caused by a virus from the Coronaviridae family.

According to an embodiment, said viral infection is caused by Severe Acute Respiratory Syndrome Coronavirus 2 (SARS-Cov-2).

According to an embodiment, said composition comprises at least one organism from each or at least two strains of the order Clostridiales. In some such embodiments, said composition comprises at least one organism from two different strains of the order Clostridiales, at least three different strains, at least four different strains or even at least five different strains.

According to an embodiment, said at least one organism has at least one property selected from the group consisting of butanoate metabolism, obligate anaerobic growth, gas fixation via the reductive acetyl-coenzyme A pathway, tolerance to bile salts at concentration greater than 0.05%, greater than 0.1% greater than 0.2% or greater than 0.4%, tolerance to pH of less than 5.5, less than 4.5 less than 3.5 or less than 2.5 and self-aggregation.

According to an embodiment, said organism comprises a member of a family selected from the group consisting of Caldicoprobacteraceae; Christensenellaceae; Clostridiaceae; Defluviitaleaceae; Eubacteriaceae; Graciibacteraceae; Heliobacteriaceae; Lachnospiraceae; Oscillospiraceae; Peptococcaceae; Peptostreptococcaceae; Ruminococcaceae; Syntrophomonadaceae and combinations thereof.

According to an embodiment, said Eubacteriaceae comprises Eubacterium aggregans.

According to an embodiment, said organism comprises a member of a genus selected from the group consisting of Acetobacterium, Acetoanaerobium, Blautia, Butyribacterium, Clostridium, Desulfitobacterium, Desulgotomaculum, Eubacterium, Hungateiclostridium, Lachnoclostridium, Moorella, Oxobacter, Paraclostridium Peptoclostridium, Pseudoclostridium, Ruminiclostridium, Sporomua, Terrisporobacter, Thermoanaerobacter, Thermoanaerobacterium, Thermoclostridium and combinations thereof.

According to an embodiment, said organism is selected from the group consisting of Acetobacterium woodii, Blautia producta, Butyribacterium methylotrophicum, Clostridium acetobutylicum, Clostridium autoethanogenum, Clostridium beijerinckii, Clostridium butyricum, Clostridium carboxidivorans, Clostridium drakei, Clostridium ljungdahlii, Clostridium kluyveri, Clostridium pasteurianum, Clostridium saccharobutylicum, Clostridium saccharoperbutylacetonicum, Clostridium scatologenes, Clostridium tyrobutyricum, Eubacterium aggregans, Eubacterium limosum, Eubacterium callendari, Eubacterium hallii, Eubacterium maltosivorans, Paraclostridium bifermentans, Oxobacter pfennigii, Sporomusa termitida, Terrisporobacter glycolicus and combinations thereof.

According to an embodiment, said composition comprises at least 10⁴ colony forming units (CFU) per milliliter of said organism, such as at least 10⁴, at least 10⁵, at least 10⁶, at least 10⁷ even at least 10⁸ CFU per milliliter of said organism, such as at least 10⁵, at least 10⁶, at least 10⁷, at least 10⁸, at least 10⁹ or up to 10¹⁰ CFU per milliliter of said organism.

According to an embodiment, said composition further comprises a carrier. According to an embodiment the carrier is selected from the group consisting of aqueous solution of salts and/or of sugars. According to an embodiment the sugars are selected from the group consisting of mannitol, lactose, cellulose, glucose, sucrose, starch, amylose, fructose, and fructose oligo saccharides (FOS). According to an embodiment the salts are selected from the group consisting of NaCl, CaCl2, MgCl2, and tris(hydroxymethyl)aminomethane. According to an embodiment the concentration of the salt ranges from 0.05% to 0.5% of the solution. According to an embodiment the concentration of the sugar ranges from 2% to 10% of the solution. According to an embodiment the carriers are selected from a group consisting of antimicrobials, antioxidants, chelating agents, inert gases, organic acids, glycol, polyethylene glycol, vegetable oils, ethyl oleate. According to an embodiment the carriers are phosphate buffer solution or glycerol solution.

According to an embodiment, said composition further comprises a reducing agent. According to an embodiment, said reducing agent is selected from the group consisting of cysteine hydrochloride, sodium sulfide, sodium sulfite, sodium metabisulfite and combinations thereof.

According to an embodiment, said composition is prepared by mixing said organism with said carrier to produce a mixture and adjusting said mixture to provide anaerobic conditions. According to an embodiment, adjusting said mixture comprises adding at least one oxygen scavenger/reducing agent, such as one selected from the group consisting of cysteine hydrochloride, sodium sulfide, sodium sulfite, sodium metabisulfite and combinations thereof.

According to an embodiment, said composition is prepared by further cooling said mixture. According to an embodiment, cooling said mixture comprises passing said mixture through cold water or a cold gas such as nitrogen.

According to an embodiment, oxygen concentration in said composition is in the range of from about 1 microMolar to about 3000 mMolar. In some embodiments the oxygen concentration is less than 100 microMolar, less than 50 microMolar, less than 10 microMolar or even less than 1 microMolar.

According to an embodiment, said composition comprises a live culture of said organism.

According to an embodiment, said composition comprises a sporulated culture of said organism.

According to an embodiment, said composition further comprises at least one selected from the group consisting of water, nutrients, prebiotics, probiotics and combinations thereof.

According to an embodiment, said composition further comprises at least one selected from the group consisting of a flavoring agent and a colorant.

According to an embodiment, said composition further comprises cells of at least one selected from the group consisting of Bacillus amyloliquefaciens; BacillusBacillus toyonensis; BacillusBacillus coagulans; BacillusBacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Eschericia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces bourlrdii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus Bovis and combinations thereof.

According to an embodiment, said composition comprises a mixture of organisms.

According to an embodiment, said mixture of organisms comprises a syntrophic mixture of organisms showing a syntrophic behavior.

According to an embodiment, said syntrophic behavior is beneficial to said animal.

According to an embodiment, said mixture of organisms comprises at least one CO₂-utilizing organism.

According to an embodiment, said CO₂-utilizing organism is an acetogen.

According to an embodiment, said acetogen is selected from the group consisting of Acetitomaculum; Acetoanaerobium; Aceto bacterium; Acetohalobium; Acetoneme; Bacillus; Blautia; Bryantella; Butyribacterium; Caloramator; Clostridium; Desulfovibrio; Entrococcus; Eubacterium; Gottschalkia; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella; and combinations thereof.

According to an embodiment, said mixture of organisms comprises at least one non-CO₂ utilizing organism.

According to an embodiment, said mixture of organisms comprises at least one acetate-forming organism and at least one acetate-utilizing organism.

According to an embodiment, said mixture of organisms comprises at least one lactate-forming organism and at least one lactate-utilizing organism.

According to an embodiment, said animal is selected from the group consisting of an avian, a human, a cat, a dog, a pig, a cow.

According to an embodiment, said animal is an avian.

According to an embodiment wherein said animal is an avian, said administering comprises applying said composition to an outer surface of an egg comprising said avian as an embryo.

According to an embodiment wherein said animal is an avian, said applying comprises spraying. According to one such embodiment, said spraying comprises electrostatic spraying.

According to an embodiment wherein said animal is an avian and said egg has a narrow end and a blunt end, said applying comprises applying to said narrow end of said egg. According to one such embodiment, an amount of said composition applied per unit area on the narrow end of the egg is greater than the amount of composition applied per unit area on the blunt end of the egg.

According to some such embodiments, an amount of said composition applied per unit area on the narrow end of the egg is greater by about 10%, at least 15%, at least 20%, at least 25%, at least 30%, at least 35%, at least 40%, at least 45%, at least 50%, at least 55%, at least 60%, at least 65%, at least 70%, at least 75% or even at least 80% greater.

According to an embodiment wherein said animal is an avian, a concentration of said organism in said composition, a temperature of said composition and an air content of said composition are each adjusted to provide at least 10² colony forming units per cm³ eggshell of said organism, such as at least at least 10², at least 10³, at least 10⁴, at least 10⁵, at least 10⁶, at least 10⁷, at least 10⁸, at least 10⁹ or even at least 10¹⁰ CFU per cm³ eggshell of said organism within said egg.

According to an embodiment wherein said animal is an avian, said applying is carried out at least twice, such as twice, three times, four times, or even five times.

According to an embodiment wherein said animal is an avian, said applying results in at least 10 organism Colony-Forming Units per gram wet feces of said avian on a second day after hatching, such as at least 10, at least 10², at least 10³, at least 10⁴, at least 10⁵, at least 10⁶, at least 10⁷, at least 10⁸, at least 10⁹ or even at least 10¹⁰ CFU per gram wet feces of said avian on a second day after hatching.

According to an embodiment wherein said animal is an avian, said applying results in at least 10² Colony-Forming Units per gram wet feces of said avian on a twentieth day after hatching, such as at least 10², at least 10³, at least 10⁴, at least 10⁵, at least 10⁶, at least 10⁷, at least 10⁸, at least 10⁹ or even at least 10¹⁰ CFU per gram wet feces of said avian on a twentieth day after hatching.

According to an embodiment, said avian is selected from the group consisting of broilers, hens, turkeys, ducks, and fowls.

According to an embodiment wherein said animal is an avian, said administering comprises in ovo injection of said composition into an egg comprising said avian as an embryo.

According to an embodiment wherein said animal is an avian, said administering comprises providing said composition to said avian orally within 2 days of hatching.

According to an embodiment wherein said animal is an avian, said providing said composition orally comprises adding said composition to a food or to a drink provided to said avian.

According to an embodiment wherein said animal is a human, said administering comprises administering orally. According to some such embodiments, administering said composition orally comprises adding said composition to a food or to a drink provided to said human. According to some such embodiments, said organism is present in said composition at a concentration of from 10 CFU to 10⁸ CFU, such as at least 10, at least 10², at least 10³, at least 10⁴, at least 10⁵, at least 10⁶, at least 10⁷, or up to 10⁸.

According to some embodiments, said composition is administered at least once a day (such as once, twice, three times, four times or more than four times a day) or at least once a week (such as once, twice, three times, four times, five times, six times or seven times per week).

According to an embodiment, upon administering of the composition as disclosed herein, the digestive track of an animal is colonized by said organism. According to an embodiment, said colonizing comprises colonizing at least one of duodenum, jejunum, ileum, small intestine, cecum, and colon.

According to one aspect of the current invention, provided is a method for preparing the composition as disclosed herein, which method comprises anaerobic fermentation, induction of sporulation and separation of the formed cells. According to an embodiment the method for induction of sporulation comprises cultivating at phosphate concentration of less than 5%, less than 3% or less than 1%. According to an embodiment, the induction of sporulation comprises cultivating at nitrogen concentration of less than 1 mM, less than 0.1 mM or less than 0.01 mM. According to an embodiment, the induction of sporulation comprises cultivating at a temperature greater than 37 degree C., greater than 55 degree C. or greater than 75 degree C. According to an embodiment, the induction of sporulation comprises cultivating at a temperature of less than 37 degree C., less than 25 degree C. or less than 10 degree C. According to an embodiment, the induction of sporulation comprises reducing or preventing respiratory problems, reducing or preventing Coccidia infection in a medium comprising at least 100 mM solvent, at least 10 mM or at least 1 mM. According to an embodiment the method for induction of sporulation comprises cultivating at carbon concentration of less than 10 mM, less than 1 mM or less than 0.1 mM. According to an embodiment the method for induction of sporulation comprises cultivating at pH of less than 6.0, less than 5.0 or less than 4.0. According to an embodiment the method for induction of sporulation comprises cultivating at pH of more than 8.0, more than 9.0 or more than 10.0.

According to an embodiment, the separating of cells, comprises at least one of flocculating, centrifuging and separating by large scale flow cytometry, to separate the vegetative cells from the spores.

According to an embodiment, said subject is an in ovo avian, wherein said method results in increased hatchability, such as increased percentage of hatched eggs and/or increased number of eggs hatched on time, wherein on time hatchability is considered as hatching on day 21 after laying.

### EXAMPLE

### In Ovo Application of Acetogens or Improvement of Hatchability Under Contaminated Conditions

The effect of in ovo feeding delivery (IOF) on hatchability under contaminated conditions was determined using a liquid composition comprising a mixture of the following 5 different species of Clostridium (CL-A1) at CFU concentrations 10¹ (Test Group I) or 10³ (Test Group II): Butyribacterium methylotrophicum; Eubacterium aggregans; Blautia producta; Clostridium carboxidivorans; and Clostridium ljungdahlii.

Each test group consisted of 87 fertile eggs.

The compositions were injected into the amnion of the eggs at day 17 after laying.

100 μL of saline (9%) was used as a control for a Control Group of 83 fertile eggs.

All eggs were maintained in a single hatcher until 21 days after laying, under optimum incubation condition.

Results are presented in Table 1 below.

As shown the table, 87% hatchability was obtained with the control, while a statistically significant increase in hatchability was obtained with each of the CL-A1 compositions (94% and 96.4% for Test Groups I and II, respectively). Furthermore, 11% of eggs of the control group hatched late (i.e. after day 21 from laying), while all eggs of Test Groups I and II hatched on time.

Without wishing to be bound by any one theory, the present Inventors hypothesize that the increased hatchability is believed to be due to a decrease in infection rate in the eggs under the contaminated conditions as compared to eggs which did not receive the CL-A1 treatment.

TABLE 1

| | % hatchability | % hatchability | % hatchability |
|---|---|---|---|
| | (on time) | (late) | (TOTAL) |
| Test Group I | 94.0% | 0% | 94.0% |
| Test Group II | 96.4% | 0% | 96.4% |
| Control Group | 76.0% | 11% | 87% |

1. A method of treating or preventing an infection in a subject in need thereof, the method comprising administering to said subject a composition comprising at least one organism of a strain of the order Clostridiales.

2. The method of claim 1, wherein said infection is selected from the group consisting of a viral infection, a protozoal infection and a fungal infection.

3. (canceled)

4. The method of claim 2, wherein said infection is a viral infection caused by Severe Acute Respiratory Syndrome Coronavirus 2 (SARS-Cov-2).

5. The method of claim 1, wherein said composition comprises at least one organism from each of at least two strains of the order Clostridiales.

6. The method of claim 1, wherein said organism has at least one property selected from the group consisting of butanoate metabolism, obligate anaerobic growth, gas fixation via the reductive acetyl-coenzyme A pathway, tolerance to bile salts at concentration greater than 0.05%, tolerance to pH of less than 3.5, and self-aggregation.

7. The method of claim 1, wherein said organism comprises a member of a family selected from the group consisting of Caldicoprobacteraceae; Christensenellaceae; Clostridiaceae; Defluviitaleaceae; Eubacteriaceae; Graciibacteraceae; Heliobacteriaceae; Lachnospiraceae; Oscillospiraceae;; Peptococcaceae; Peptostreptococcaceae; Ruminococcaceae; Syntrophomonadaceae and combinations thereof, optionally wherein said Eubacteriaceae comprises Eubacterium aggregans.

8. (canceled)

9. The method of claim 1, wherein said organism comprises a member of a genus selected from the group consisting of Acetobacterium, Acetoanaerobium, Blautia, Butyribacterium, Clostridium, Desulfitobacterium, Desulgotomaculum, Eubacterium, Hungateiclostridium, Lachnoclostridium, Moorella, Oxobacter, Paraclostridium Peptoclostridium, Pseudoclostridium, Ruminiclostridium, Sporomua, Terrisporobacter, Thermoanaerobacter, Thermoanaerobacterium, Thermoclostridium and combinations thereof.

10. The method of claim 1, wherein said organism is selected from the group consisting of Acetobacterium woodii, Blautia producta, Butyribacterium methylotrophicum, Clostridium acetobutylicum, Clostridium autoethanogenum, Clostridium beijerinckii, Clostridium butyricum, Clostridium carboxidivorans, Clostridium drakei, Clostridium ljungdahlii, Clostridium kluyveri, Clostridium pasteurianum, Clostridium saccharobutylicum, Clostridium saccharoperbutylacetonicum, Clostridium scatologenes, Clostridium tyrobutyricum, Eubacterium aggregans, Eubacterium limosum, Eubacterium callendari, Eubacterium hallii, Eubacterium maltosivorans, Paraclostridium bifermentans, Oxobacter pfennigii, Sporomusa termitida, Terrisporobacter glycolicus and combinations thereof.

11-12. (canceled)

13. The method of claim 1, wherein said composition further comprises a reducing agent.

14-19. (cancelled).

20. The method of claim 1, wherein said composition further comprises cells of at least one selected from the group consisting of Bacillus amyloliquefaciens; BacillusBacillus toyonensis; BacillusBacillus coagulans; BacillusBacillus licheniformis; Bacillus megaterium; Bacillus mesentricus; Bacillus polymyxa; Bacillus subtilis; Bifidobacterium animalis; Bifidobacterium bifidium; Bifidobacterium bifidus; Bifidobacterium thermophilus; Bifidobacterium longum; Bifidobacterium pseudolongum; Bifidobacterium lactis; Clostridium butyricum; Enterococcus faecium; Enterococcus faecalis; Eschericia coli; Lactobacillus thermophilus; Lactobacillus acidophilus; Lactobacillus brevis; Lactobacillus bulgaricus; Lactobacillus casei; Lactobacillus delbrueckii; Lactobacillus subspecies; Lactobacillus bulgaricus; Lactobacillus farciminis; Lactobacillus fermentum; Lactobacillus gallinarum; Lactobacillus jensenii; Lactobacillus paracasei; Lactobacillus plantarum; Lactobacillus reuteri; Lactobacillus rhamnosus; Lactobacillus lactis; Lactobacillus salivarius; Lactobacillus sobrius; Megasphaera elsdenii; Pediococcus acidolactici; Propionibacterium shermanii; Propionibacterium freudenreichii; Propionibacterium acidipropionici; Propionibacterium jensenii; Saccharomyces bourlrdii; Saccharomyces cerevisiae; Saccharomyces servisia; Streptococcus faecalis; Streptococcus faecium; Streptococcus gallolyticus; Streptococcus salivarius; Streptococcus subsp.; Streptococcus thermophilus; Streptococcus bovis and combinations thereof.

21-23. (canceled)

24. The method of claim 1, wherein said mixture of composition comprises at least one CO₂-utilizing organism, optionally wherein said CO₂-utilizing organism is an acetogen.

25. (canceled)

26. The method of claim 24, wherein said acetogen is selected from the group consisting of Acetitomaculum; Acetoanaerobium; Acetobacterium; Acetohalobium; Acetoneme; Bacillus; Blautia; Bryantella; Butyribacterium; Caloramator; Clostridium; Desulfovibrio; Entrococcus; Eubacterium; Gottschalkia ; Holophage; Methylobacterium; Micrococcus; Moorella; Mycobacterium; Natronielle; Natronincola; Oxobacter; Peptoniphilus; Proteus; Reticulitermes; Rhizobium; Ruminococcus; Saccharomyces; Sinorhizobium; Sphingomonas; Sporomusa; Syntrophococcus; Thermoacetogenium; Tindallia; Treponema; Veillonella and combinations thereof.

27. The method of claim 1, wherein said composition comprises at least one non-CO₂ utilizing organism.

28. The method of claim 1, wherein said composition comprises at least one acetate-forming organism and at least one acetate-utilizing organism.

29. The method of claim 1, wherein said composition comprises at least one lactate-forming organism and at least one lactate-utilizing organism.

30. The method of claim 1, wherein said animal is selected from the group consisting of an avian, a human, a cat, a dog, a pig, a cow.

31. (canceled)

32. The method of claim 30, wherein said animal is an avian and wherein said administering comprises applying said composition to an outer surface of an egg comprising said avian as an embryo.

33. The method of claim 32, wherein said applying comprises spraying, optionally electrostatic spraying.

34. (canceled)

35. The method of claim 32, said egg having a narrow end and a blunt end, wherein said applying comprises applying to said narrow end of said egg.

36-41. (canceled)

42. The method of claim 30, wherein said animal is an avian and wherein said administering comprises in ovo injection of said composition into an egg comprising said avian as an embryo.

43. The method of claim 30, wherein said animal is an avian and wherein said administering comprises providing said composition to said avian orally within 2 days of hatching.

44. (canceled)

45. The method of claim 30, wherein said animal is a human, wherein said administering comprises administering orally.

46-48. (canceled)
